(19) United States
(12) Patent Application Publication
(10) Pub. No.: US 2005/0220755 A1
(43) Pub. Date: Oct. 6, 2005

(54) Topical composition based on ion-exchange resins, in particular for treating erythemas

(76) Inventor: Jack Auzerie, Sainte-Eulalie (FR)

Correspondence Address: YOUNG & THOMPSON, 745 SOUTH 23RD STREET, 2ND FLOOR, ARLINGTON, VA, 22202, US

(21) Appl. No.: 10/509,388

(22) PCT Filed: Mar. 26, 2003
(86) PCT No.: PCT/FR03/00948

§ 371 Date: Sep. 27, 2004

(30) Foreign Application Priority Data
Mar. 26, 2002 (FR) 02/03729

(51) Int. Cl. A61K 31/785

(52) U.S. Cl. 424/78.12

(57) ABSTRACT

The object of the invention is a topical composition particularly for the treatment of erythema, characterized in that it includes at least one ion exchange resin such as colestyramine and at least one substance with buffer properties.

The present invention relates to a topical composition based on ion exchange resins such as colestyramine, particularly for the treatment of erythema of the buttocks in pediatrics.

Acute diarrhea affects numerous children, particularly children less than five years old, often requiring treatment to avoid secondary infection and to relief children from the pain and itching associated therewith.

Thus, episodes of acute diarrhea give rise to dermatological problems, particularly cutaneous lesions difficult to heal.

One of the factors which initiates this type of dermatological problem is the defecation of bile salts which are then in intimate contact with the skin.

First of all, these bile salts which are acidic, lower the cutaneous pH and give rise to erythema.

Then, these bile salts have tensioactive properties, so that the sebaceous layer which is naturally protective, is eliminated.

To avoid these afflictions, one can use:
1/ on the one hand, with oral treatment, particularly the taking of:
   antibiotics,
   lactic ferments,
   optiates, particularly loperamide,
   active principles with anti-secretive activity, such as the commercial product Tiorfan, and
   clays, sold under the names Smecta or Actapulgite.
2/ on the other hand with the application of a cream or pommade, particlarly
   those known by the commercial names Jonctum, Madécassol, Mytosil,
   these including dermocorticoids associated or not with antibiotics.

Such preparations are said to have trophic or protective activity.

The results are hardly satisfying because the occlusive barrier created can take place in secondary infection of the sore by the appearance of anaerobic bacterial flora.

Moreover, these preparations do not neutralize the acidity of the bile salts.

These known treatments thus do not give satisfaction.

Particularly colestyramine is a basic synthetic ion exchange resin which has a high affinity for bile salts and forms with them an insoluble complex. It is known that 1 gram of colestyramine can absorb up to two grams of bile salts. It is a fine hygroscopic powder, white in color, substantially insoluble in water, alcohol, chloroform and ether. In a 1% solution, the pH is between 4 and 6.

A medication whose commercial name is Questran, is indicated for the treatment of hypercholestive rolemia.

This powder permits adsorbing biosalts present in the gastrointestinal tract. One can thus prevent the formation of calculi and decrease the circulating triglycerides.

The present invention relates to a composition including at least one ion exchange resin, particularly colestyramine as well as a pH regulator.

Thus, in an entirely pertinent manner, the regulator permits increasing the pH to permit optimum absorption of the bile salts by the ion exchange resins.

Thus a pH comprised between 4 and 8, preferably of about 6, is provided.

The composition according to the invention will now be described with the help of particular non-limiting examples.

A basic composition according to the present invention comprises the following elements:
30% by weight of colestyramine,
30% by weight of aluminum hydroxide,
30% by weight of magnesium hydroxide, and
10% of water

This composition produces a thick paste.

Tests have been conducted on subjects afflicted with secondary dermatitis:
with gastroenteritis,
with small intestine syndrome, and
with neonatal necrotic enterocolitis.

The thick paste of colestyramine base is applied 4 times per day to the region corresponding substantially to the region covered by a diaper.

Medical observation is conducted upon each application.

There is noted a regression of the lesions at the end of 2 to 7 days.

Thus the composition permits local physiological rebalancing upon the appearance of irritating dermatitis.

The pH regulators can also be selected from: monopotassium phosphate, disodium phosphate, sodium acetate, ammonium acetate, sodium citrate, citric acid, disodium tetraborate, boric acid, amino acids such as lysine, leucine, alanine, methyl parahydroxybenzoate, propyl parahydroxybenzoate, sodium benzoate.

More generally, substances can be used that have buffer properties.

Examples of galenic formulations in the form of creams or powders are indicated hereafter.

1/ Cream with Variable Proportions of Colestyramine:

| | | Quantity | Quantity | Quantity |
|---|---|---|---|---|
| COMPONENTS | FUNCTION | % | % | % |
| Colestyramine | Adsorbent | 5.000 | 10.000 | 15.000 |
| Labrafil M1944C | Oily phase | 3.000 | 3.000 | 3.000 |
| White vaseline | Oily phase | 3.000 | 3.000 | 3.000 |
| Tefose 63 | Tensioactive | 10.000 | 10.000 | 10.000 |
| Butylhydroxyanisol | Antioxidant | 0.005 | 0.005 | 0.005 |
| Methyl | Preservative | 0.100 | 0.100 | 0.100 |
| parahydroxybenzoate | | | | |
| Propyl | Preservative | 0.050 | 0.050 | 0.050 |
| parahydroxybenzoate | | | | |
| Honeysuckle perfume | Perfume | 0.500 | 0.500 | 0.500 |
| Water adjusted to | pH regulator | 78.345 | 73.345 | 68.345 |
| pH 9 with NaOH | | | | |
| 1 N | | | | |

| | | | |
|---|---|---|---|
| TOTAL | 100.000 | 100.000 | 100.000 |

Labrafil: commercial name of oleic macrogoglycerides
Tefose 63: commercial name of PEG-6-32 Stearate and glycol stearate.

2/ Cream with 10% of Colestyramine and Sodium Dihydrogenophosphate:

| | | Quantity |
|---|---|---|
| COMPONENTS | FUNCTION | % |
| Colestyramine | Adsorbent | 10.000 |
| White vaseline | Oily phase | 3.000 |
| Labrafil M1944C | Oily phase | 3.000 |
| Tefose 63 | Tensioactive | 10.000 |
| Butylhydroxyanisol | Antioxidant | 0.005 |
| Methyl | Preservative | 0.100 |
| parahydroxybenzoate | | |
| Propyl | Preservative | 0.050 |
| parahydroxybenzoate | | |
| Honeysuckle perfume | Perfume | 1.000 |
| Sodium | pH regulator | 1.000 |
| dihydrogenophosphate | | |
| Water | Aqueous | 71.845 |
| | phase | |

| | |
|---|---|
| TOTAL | 100.000 |

3/ Talc Comprising 10% Colestyramine:

| COMPONENTS | FUNCTION | Quantity % |
|---|---|---|
| Colestyramine | Adsorbent | 10 |
| Sodium | pH regulator | 1 |
| dihydrogenophosphate | | |
| Talc | Dilutant | 89 |

| | |
|---|---|
| TOTAL | 100 |

There can be provided different galenic forms:
cream,
gels,
lotions,
aerosols,
spray aerosols

The composition can also be provided for direct application on external supports in direct contact with the zones to be treated such as the diapers for nursing babies, children and aged persons.

1. Topical composition particularly for the treatment of erythema, characterized in that it includes at least colestyramine as an ion exchange resin and at least one substance that has buffer properties.

2. Topical composition according to claim 1, characterized in that the substance with buffer properties is selected from monopotassium phosphate, disodium phosphate, sodium acetate, ammonium acetate, sodium citrate, citric acid, disodium tetraborate, boric acid, amino acids such as lysine, leucine, alanine, methyl parahydroxybenzoate, propyl parahydroxybenzoate, sodium benzoate.

3. Topical composition according to claim 1, characterized in that it comprises a pH regulator comprising imparting to said composition a pH between 4 and 8, to permit optimum absorption of the bile salts by the ion exchange resins.

4. Topical composition according to claim 3, characterized in that the pH regulator permits imparting to said composition a pH of about 6.0.

5. Topical composition according to claim 1, characterized in that it is present in one of the different galenic forms: cream, gels, lotions, aerosols or spray aerosols.

6. Topical composition according to claim 1, characterized in that it is applied directly to external supports in direct contact with the zones to be treated.

7. Topical composition according to claim 2, characterized in that it comprises a pH regulator comprising imparting to said composition a pH between 4 and 8, to permit optimum absorption of the bile salts by the ion exchange resins.
